(19) United States
(12) Patent Application Publication
(10) Pub. No.: US 2017/0072130 A1
(43) Pub. Date: Mar. 16, 2017

(54) COMPACT KIT FOR INJECTING LIQUID MEDICATION

(71) Applicant: Douglas F. MCMAHON, Woodbury, MN (US)

(72) Inventor: Douglas F. MCMAHON, Woodbury, MN (US)

(21) Appl. No.: 15/126,599

(22) PCT Filed: Mar. 17, 2015
(86) PCT No.: PCT/US2015/021001

§ 371 Date: Sep. 16, 2016

Related U.S. Application Data
(60) Provisional application No. 61/954,922, filed on Mar. 18, 2014.

(51) Int. Cl. A61M 5/00; A61M 5/32; A61M 5/315; A61M 5/34; A61M 5/28

(52) U.S. Cl. CPC A61M 5/002; A61M 5/345; A61M 5/28; A61M 5/31511; A61M 5/3202

(57) ABSTRACT

To administrate a liquid medication in an emergency situation, an injection kit includes a syringe assembly, a needle assembly, and a container. The container contains the syringe assembly and the needle assembly, and possessed and carried closely by a patient. When an immediate injection of the liquid medication to the patient is needed, the syringe assembly and the needle assembly are taken out from the container and assembled by the patient or a bystander.

This application is being filed on 17 Mar. 2015, as a PCT International patent application, and claims priority to U.S. Provisional Patent Application No. 61/954,922, filed Mar. 18, 2014, the disclosure of which is hereby incorporated by reference herein in its entirety.

### BACKGROUND

Anaphylaxis is a life-threatening allergic reaction. The symptoms of such allergic reactions include severe swelling, breathing problems, or loss of blood pressure. The allergic reactions can be caused by stringing and biting insects, allergy injections, food, medicines, exercise, or unknown causes.

The anaphylaxis is rapid in onset and may cause death. Thus, emergency treatment is necessary before going to doctor or emergency room for more medical treatment. The primary emergency treatment is an injection of epinephrine.

Several types of epinephrine injection devices are used for emergency administration of epinephrine. An example of such injection devices is an epinephrine auto-injector, as shown in FIG. 1. Typically, a user, either a patient or a spectator, puts a tip of the injector against the middle of the outer side of the patient's upper leg, presses down hard until the needle enters the upper leg through the skin, and holds it in place for a predetermined amount of time. Then, the injector is removed from the upper leg. The remainder of the epinephrine needs to be carefully discarded.

### SUMMARY

In general terms, this disclosure is directed to a medication injection kit. In one possible configuration and by non-limiting example, the injection kit includes an injection system contained in a compact-sized container. Various aspects are described in this disclosure, which include, but are not limited to, the following aspects.

One aspect is a system for administrating a liquid medication in an emergency situation, the system comprising: a syringe assembly comprising: a barrel portion containing the liquid medication; a plunger portion sealingly engaged with the barrel portion to form a chamber for expelling the liquid medication from the barrel portion; and a needle adaptor, a needle assembly comprising: a needle hub having a needle end and a coupling end; a needle shaft extending from the needle end of the needle hub; a plug portion arranged at the coupling end of the needle hub and configured to engage the needle adaptor; and a protective needle cover configured to at least partially cover the needle hub and the needle shaft, and a container configured to contain the syringe assembly and the needle assembly, the container being possessed and carried by a patient, wherein, in the emergency situation, the syringe assembly and the needle assembly are taken out from the container and assembled by coupling the plug portion to the needle adaptor such that the needle shaft is in fluid communication with the chamber.

Another aspect is a method for administering a liquid medication in an emergency situation, the method comprising: placing a syringe assembly and a needle assembly within a container; and providing a coupling mechanism for coupling the container to a coupling arrangement closely possessed by a patient, wherein the patient or a bystander takes out the syringe assembly and the needle assembly, assemble the syringe assembly and the needle assembly, and administer the liquid medication to the patient.

### BRIEF DESCRIPTION OF THE DRAWINGS

FIG. 1 shows an example of a prior art auto-injector for administering epinephrine.

FIG. 2 is a schematic view of an example kit for injecting liquid medication.

FIG. 3 is a side perspective view of an example syringe assembly.

FIG. 4 is a front perspective view of an example needle adaptor of the syringe assembly of FIG. 3.

FIG. 5 is a side perspective view of an example needle assembly.

FIG. 6 is a side perspective view of the needle assembly of FIG. 5 with a protective needle cover removed.

FIG. 7 is a side perspective view of the injection system of FIG. 2, illustrating that the syringe assembly and the needle assembly have been coupled.

FIG. 8 is a side perspective view of the injection system of FIG. 2, illustrating that the protective needle cover is removed after the syringe assembly and the needle assembly is assembled.

FIG. 9 is a schematic view of another example container.

FIG. 10 is a side view of an example kit for injecting liquid medication.

FIG. 11 is an exploded view of the example kit of FIG. 10.

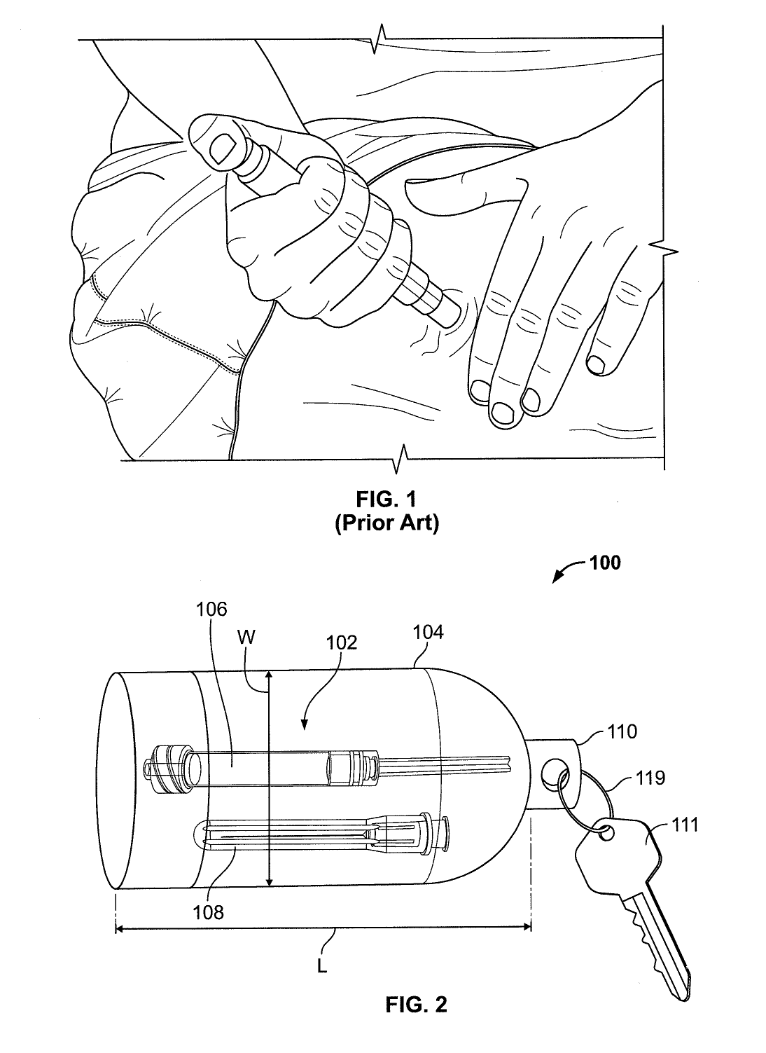

### DETAILED DESCRIPTION

Various embodiments will be described in detail with reference to the drawings, wherein like reference numerals represent like parts and assemblies throughout the several views. Reference to various embodiments does not limit the scope of the claims attached hereto. Additionally, any examples set forth in this specification are not intended to be limiting and merely set forth some of the many possible embodiments for the appended claims.

FIG. 2 is a schematic view of an example kit 100 for injecting liquid medication. In some embodiments, the kit 100 is used in an emergency situation where a patient needs to be treated with liquid medication before delivered to an emergency room. In at least one embodiment, the kit 100 is configured to inject epinephrine into a patient who suddenly shows a life-threatening allergic reaction. In other embodiments, however, the kit 100 is used to inject other types of liquid medication to a patient as a first aid or emergency treatment.

In some embodiments, the kit 100 includes an injection system 102 and a container 104.

The injection system 102 is configured to inject liquid medication to a patient. In some embodiments, the injection system 102 is operated by a patient or an assistant, who can be a parent, medical practitioner, or bystander. The injection system 102 is configured for convenient possession, carrying, and handling. Further, the injection system 102 is dimensioned to contain only one dose of liquid medication.

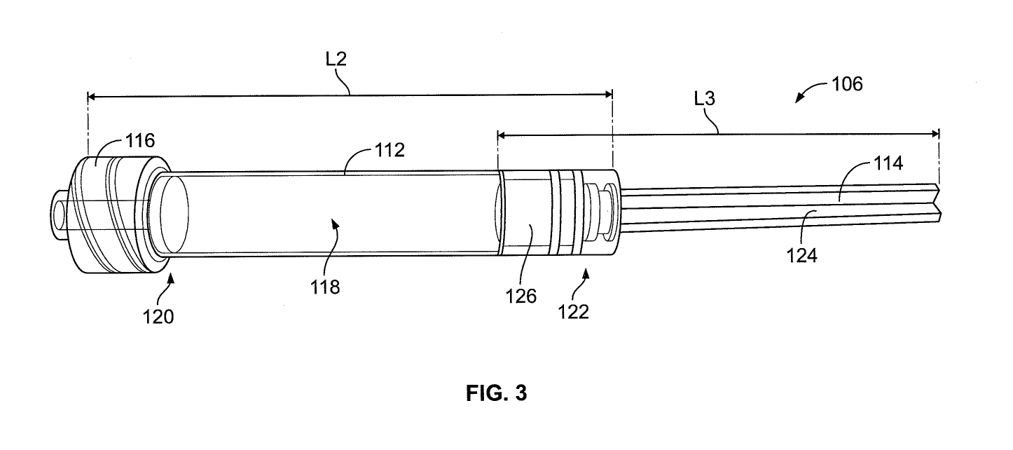

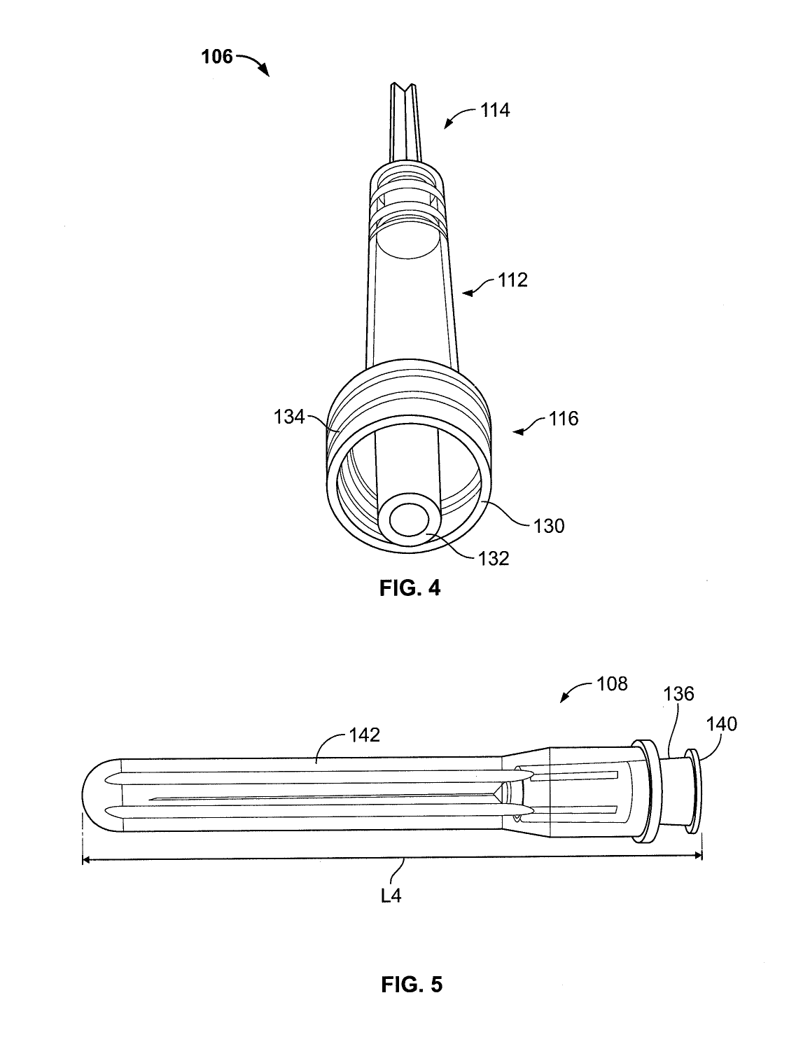

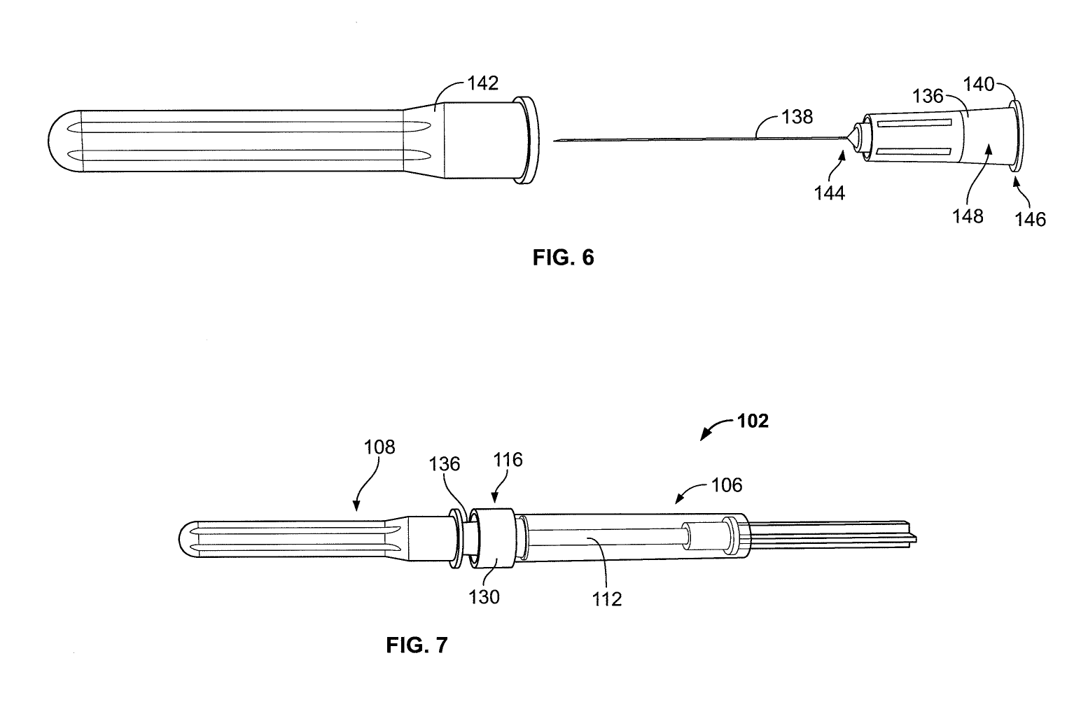

In some embodiments, the injection system 102 includes a syringe assembly 106 and a needle assembly 108. The syringe assembly 106 is described in further detail with reference to FIGS. 3 and 4. The needle assembly 108 is described in further detail with reference to FIGS. 5 and 6.

The container 104 is configured to contain the injection system 102 therein so that the injection system 102 is conveniently possessed and carried by a patient or any other people who take care of the patient. In some embodiments, the container 104 is only as much sized to include the injection system 102 and small enough to permit a child to easily carry the kit 100 so that, when the child suddenly suffers from anaphylaxis, anybody around the child can take the kit 100 from the child and administer liquid medication (for example, epinephrine) to the child by operating the injection system 102. In some embodiments, the kit 100 is coupled to any coupling arrangement closely possessed by a patient, such as a necklace worn by a patient, or to a belt loop of pants worn by the patient, so that the kit 100 is not lost and easily found by a spectator or bystander who witnesses sudden life-threatening symptoms of the patient. In some embodiments, the container 104 has a length (L) of 50-100 mm, and a diameter or width (W) of 10-40 mm. In other embodiments, the length (L) of the container 104 ranges between 65 and 80 mm, and the diameter or width (W) ranges between 20 and 25 mm. In yet other embodiments, the length (L) of the container ranges from 50-1000 mm and the diameter or width (W) ranges from 10-300 mm. In one implementation, the length (L) is approximately 889 mm and the width (W) is approximately 254 mm, although many other dimensions are possible.

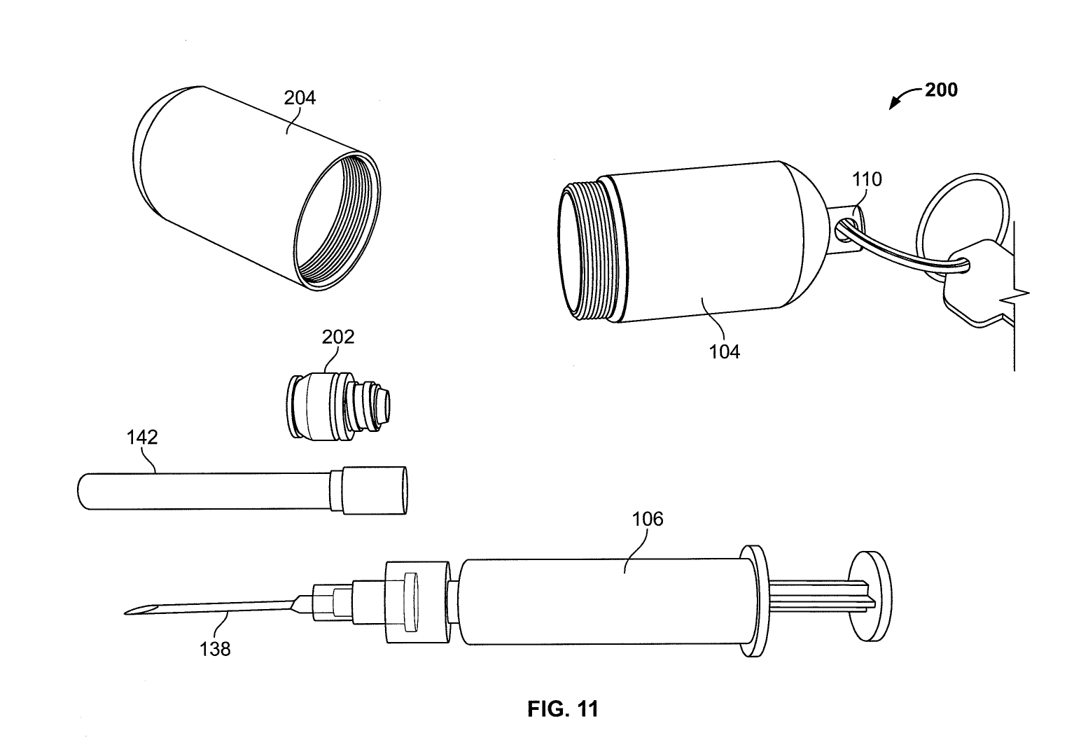

The container 104 can have a variety of shapes. In some embodiments, the container 104 has a cylindrical vessel. In other embodiments, the container 104 has a capsule shape. In yet other embodiments, the container 104 is made as a pouch. The container 104 is made from a variety of materials. In some embodiments, the container 104 is made from metal, plastic, or fabric. Further, the container 104 is configured to be fastened in various manners. In some embodiments, the container 104 has an outer surface made from rigid materials for protecting the injection system 102 contained therein. In addition, the container 104 includes an inner surface made from cushion materials for further protection of the injection system 102. In some embodiments, the container 104 can be opened or closed with a hook-and-loop fastener, such as Velcro. In other embodiments, the container 104 is fastened with a screw cap arranged at one end of the container 104. See screw cap 204 of the example kit 200 shown in FIGS. 10-11.

In some embodiments, the container 104 includes a coupling mechanism for coupling the container 104 to a coupling arrangement closely possessed by a patient. Examples of the coupling mechanism include a hook or ring 110 configured to be coupled to a keychain, a belt loop, or any other coupling arrangements. For example, the hook or ring 110 is coupled to a necklace worn by a child, or coupled to a belt loop of pants worn by a child. In other examples, the hook or ring 110 is coupled to one or more keys 111 through a keychain 109.

FIG. 3 is a side perspective view of an example syringe assembly 106. The syringe assembly 106 includes a barrel portion 112, a plunger portion 114, and a needle adaptor 116.

In some embodiments, the barrel portion 112 has an elongate bore 118 extending between a first end 120 and a second end 122 along a longitudinal axis. The elongate bore 118 is configured to contain liquid medication therein. In some embodiments, the barrel portion 112 is formed from thermoplastic materials such as polypropylene, polyethylene, polycarbonate and copolymers or any other material suitable for the barrel portion 112.

In some embodiments, the plunger portion 114 includes an elongate plunger body 124 and a gasket 126. The plunger body 124 is sized to fit slidably within the elongate bore 118 of the barrel portion 112 by advancing the plunger body 124 into the first end 120 of the barrel portion 112. The gasket 126 is mounted at a head end of the plunger body 124 for occluding the head end of the plunger body 124 and forming a slidable seal with the elongate bore 118 of the barrel portion 112 to define a chamber for drawing and expelling liquid medication from the barrel portion 112. In some embodiments, the plunger body 124 is formed from polypropylene, polyethylene, polystyrene, or any other material suitable for the plunger body 124.

In some embodiments, the needle adaptor 116 is configured to engage the needle assembly 108 therein. The needle adaptor 116 is arranged at the first end 120 of the barrel portion 112. The needle adaptor 116 is described in further detail with reference to FIG. 4.

In some embodiments, the syringe assembly 106 is configured to contain 0.3 cc of liquid medication (for example, epinephrine). This is because, for epinephrine injection, many patients only need 0.3 cc or less of epinephrine. An excessive injection of epinephrine can cause dangerously high blood pressure, stroke, or death, and thus, it is very important to limit a dosage of epinephrine, as described herein. In other embodiments, the syringe assembly 106 is configured to contain the liquid medication of more than 0.3 cc but not more than 0.5 cc. In yet other embodiments, the syringe assembly 106 is configured to contain the liquid medication of less than 0.3 cc.

To achieve a compact injection system 102, the barrel portion 112 has a length (L2) of 25-50 mm, and the plunger portion 114 has a length (L3) of 10-30 mm. In other embodiments, the length (L2) of the barrel portion 112 is limited to about 35 mm, and the length (L3) of the plunger portion 114 is limited to about 20 mm. In yet other embodiments, the length (L2) ranges from 25-600 mm and the length (L3) ranges from 10-100 mm. In one implementation, the length (L2) is approximately 506 mm and the length (L3) is approximately 50 mm. Other varying dimensions are possible.

FIG. 4 is a front perspective view of an example needle adaptor 116 of the syringe assembly 106 of FIG. 3. In some embodiments, the needle adaptor 116 includes a coupling head portion 130 and an outlet portion 132.

The coupling head portion 130 is arranged at the first end 120 of the barrel portion 112 and configured to engage the needle assembly 108. In some embodiments, the coupling head portion 130 includes a threaded portion 134 formed on an inner surface of the coupling head portion 130. As shown below, the threaded portion 134 is configured to engage a plug portion of the needle assembly 108.

The outlet portion 132 operates to provide a flow channel of liquid medication from the barrel portion 112 to the needle assembly 108. In some embodiments, the outlet portion 132 extends from the first end 120 of the barrel portion 112 and surrounded by the coupling head portion 130, as depicted in FIG. 4. As shown below, the outlet portion 132 is configured to engage a hub hollow of the needle assembly 108 so as to provide a fluid communication between the barrel portion 112 and a needle shaft of the needle assembly 108.

In some embodiments, the needle adaptor 116 is covered by a shield cap (see shield cap 202 in FIGS. 10-11) so that the syringe assembly 106 is sealed before it is assembled with the needle assembly 108 for injecting liquid medication to a patient. When in used, the shield cap is removed from the needle adaptor 116 to open the coupling head portion 130 and the outlet portion 132 to engage the needle assembly 108. The shield cap can be configured to have a height of about 3 mm to achieve a compact syringe assembly 106. In other embodiments, any type of sealing devices is used to seal the needle adaptor 116 until the injection system 102 is assembled and used. In yet other embodiments, the needle adaptor 116 need not be covered or sealed because of sufficient differential pressure inside and outside the barrel portion 112 and/or sufficient surface tension of the liquid medication contained in the barrel portion 112 at the first end 120 thereof.

FIGS. 5 and 6 illustrate an example needle assembly 108 of the injection system 102 of FIG. 2. In particular, FIG. 5 is a side perspective view of an example needle assembly 108, and FIG. 6 is a side perspective view of the needle assembly 108 of FIG. 5 with a protective needle cover removed. In some embodiments, the needle assembly 108 includes a needle hub 136, a needle shaft 138, a plug portion 140, and a protective needle cover 142.

The needle hub 136 is configured to hold the needle shaft 138 and be engaged with the needle adaptor 116. In some embodiments, the needle hub 136 has a needle end 144 and a coupling end 146 along the longitudinal axis, and includes a hub hollow 148 extending between the needle end 144 and the coupling end 146 within the needle hub 136. The hub hollow 146 is configured to engage the outlet portion 132 of the needle adaptor 116 to provide fluid communication from the barrel portion 112 to the needle shaft 138 through the outlet portion 132 when the needle assembly 108 is assembled with the syringe assembly 106.

The needle shaft 138 extends from the needle end 144 of the needle hub 136 and becomes in fluid communication with the barrel portion 112 when the needle assembly 108 is coupled to the syringe assembly 106.

The plug portion 140 is arranged at the coupling end 146 of the needle hub 136 and configured to be engaged with the coupling head portion 130 of the syringe assembly 106. In some embodiments, the plug portion 140 is formed as a flange extending from the circumference of the needle hub 136 at the coupling end 146, and configured to be screwed into the threaded portion 134 of the coupling head portion 130.

The protective needle cover 142 operates to at least partially cover the needle hub 136 and the needle shaft 138 to protect the needle shaft 138 before the needle assembly 108 is coupled to the syringe assembly 106.

In some embodiments, the needle assembly 108 with the protective needle cover 142 engaged has a length (L4) of 40-55 mm to achieve a compact injection kit 100. In other embodiments, the length (L4) of the needle assembly 108 is about 48 mm.

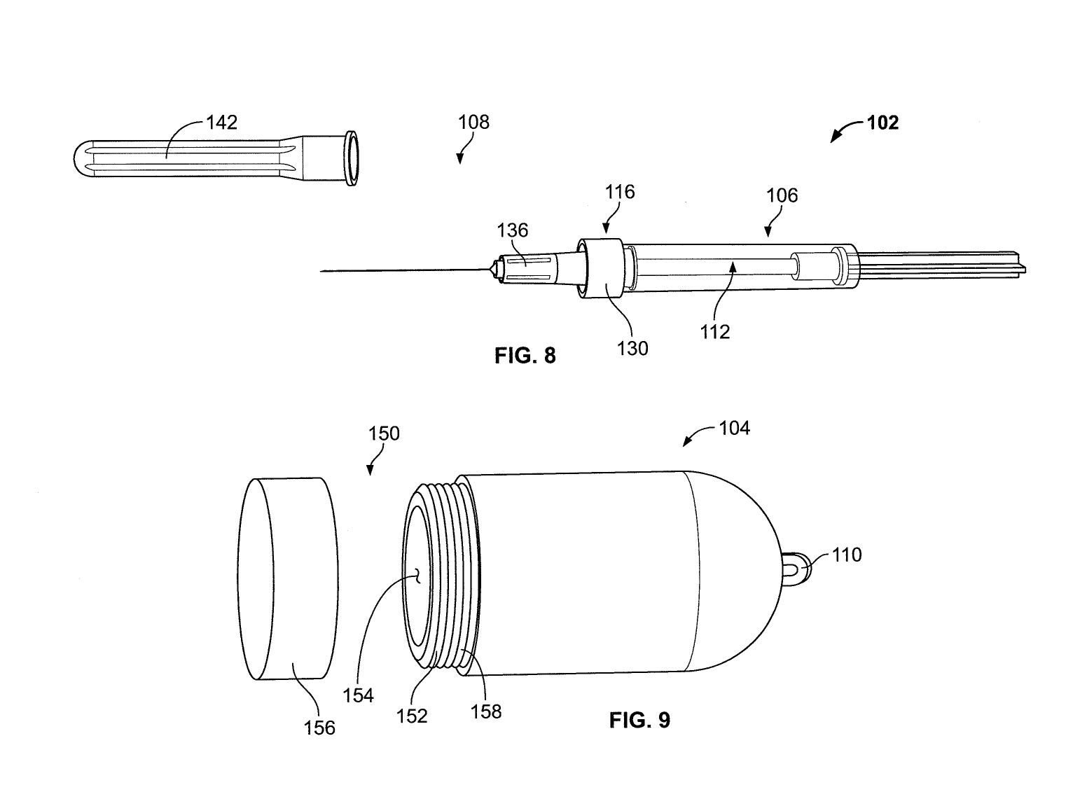

FIGS. 7 and 8 illustrate that the syringe assembly 106 and the needle assembly 108 have been coupled. In particular, FIG. 7 is a side perspective view of the injection system 102 of FIG. 2, illustrating that the syringe assembly 106 and the needle assembly 108 have been coupled. FIG. 8 is a side perspective view of the injection system 102 of FIG. 2, illustrating that the protective needle cover 142 is removed after the syringe assembly 106 and the needle assembly 108 is assembled. As depicted, the needle hub 136 is inserted to, and engaged with, the needle adaptor 116 by screwing the plug portion 140 of the needle assembly 108 into the threaded portion 134 of the coupling head portion 130. In this configuration, the needle shaft 138 is in fluid communication with the barrel portion 112 through the outlet portion 132. Then, a user can inject the medication by applying a force to the plunger portion 114 to move the plunger portion 114 from the second end 122 to the first end 120 within the elongate bore 118 along the longitudinal axis.

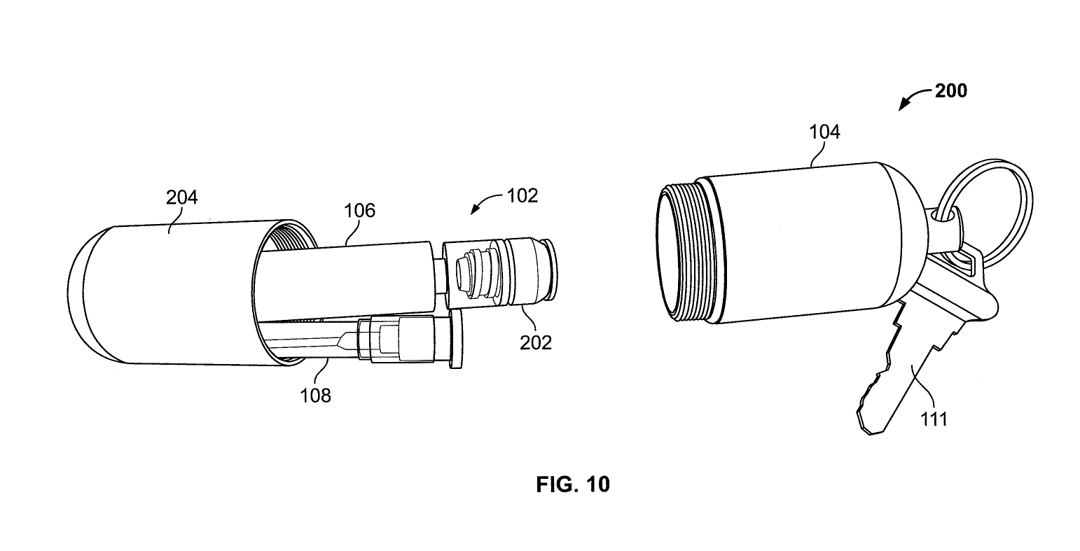

FIG. 9 is a schematic view of another example container 104. As many of the concepts and features are similar to the first example container 104 shown in FIG. 2, the description for the first example is hereby incorporated by reference for this example. Where like or similar features or elements are shown, the same reference numbers will be used where possible. The following description for this example will be limited primarily to the differences from the first example.

In the depicted example, the container 104 further includes a medication container 150. The medication container 150 is configured to contain additional medication. In some embodiments, such additional medication is suitable for relieve the symptoms of a patient in addition to the liquid medication injected to the patient by the injection system 102. For example, in addition to epinephrine, diphenhydramine, such as Benadryl, can be contained in the medication container 150 for further relief of sudden allergic reactions.

In some embodiments, the medication container 150 includes an elevated portion 152 extending from a bottom end of the container 104. The elevated portion 152 is configured to have a hollow 154 therein to provide a space for storing additional medicines. The medication container 150 further includes a cap 156 to open and close the hollow 154. The cap 156 can fit to the elevated portion 152 in various manners. In some embodiments, the elevated portion 152 includes an externally threaded portion 158 configured to engage the cap 156.

The various embodiments described above are provided by way of illustration only and should not be construed to limit the claims attached hereto. Those skilled in the art will readily recognize various modifications and changes that may be made without following the example embodiments and applications illustrated and described herein, and without departing from the true spirit and scope of the following claims.

What is claimed is:

1. A system for administrating a liquid medication, the system comprising:
a syringe assembly comprising:
a barrel portion containing the liquid medication;
a plunger portion sealingly engaged with the barrel portion to form a chamber for expelling the liquid medication from the barrel portion; and
a needle adaptor,
a needle assembly comprising:
a needle hub having a needle end and a coupling end;
a needle shaft extending from the needle end of the needle hub;
a plug portion arranged at the coupling end of the needle hub and configured to engage the needle adaptor; and
a protective needle cover configured to at least partially cover the needle hub and the needle shaft, and
a container configured to contain the syringe assembly and the needle assembly, the container being possessed and carried by a patient,
wherein the syringe assembly and the needle assembly are assembled by coupling the plug portion to the needle adaptor such that the needle shaft is in fluid communication with the chamber.

2. The system of claim 1, wherein the liquid mediation is epinephrine.

3. The system of claim 1, wherein the container includes a hook configured to be coupled to a coupling arrangement possessed by the patient.

4. The system of claim 1, wherein the container has a length of 65-80 mm and a diameter or width of 20-25 mm.

5. The system of claim 2, wherein the syringe assembly contains about 0.3 cc of the liquid medication.

6. The system of claim 5, wherein the barrel portion has a length of about 35 mm, and wherein the plunger portion has a length of about 20 mm.

7. A method for administering a liquid medication in an emergency situation, the method comprising:
placing a syringe assembly and a needle assembly within a container;
providing a coupling mechanism for coupling the container to a coupling arrangement;
taking out the syringe assembly and the needle assembly;
assembling the syringe assembly and the needle assembly; and
administering the liquid medication to the patient.

8. The method of claim 7, wherein the liquid mediation is epinephrine.

9. The system of claim 7, wherein the container includes a hook configured to be coupled to a coupling arrangement possessed by the patient.

10. The system of claim 7, wherein the container has a length of 50-1000 mm and a diameter or width of 10-300 mm.

11. The system of claim 8, wherein the syringe assembly contains about 0.3 cc of the liquid medication.

12. The system of claim 11, wherein the barrel portion has a length of about 506 mm, and wherein the plunger portion has a length of about 50 mm.
